(19) United States
(12) Patent Application Publication
(10) Pub. No.: US 2026/0115498 A1
(43) Pub. Date: Apr. 30, 2026

(54) ULTRASOUND TREATMENT DEVICE

(71) Applicant: EDGECARE INC., Seoul (KR)

(72) Inventors: Ji Hun JANG, Seoul (KR); Jung Jun KIM, Seoul (KR); Yang Mo YOO, Seoul (KR)

(21) Appl. No.: 19/316,047

(22) Filed: Sep. 2, 2025

(30) Foreign Application Priority Data
Oct. 30, 2024 (KR) 10-2024-0150628

(51) Int. Cl. A61N 7/00

(52) U.S. Cl. CPC A61N 7/00; A61N 2007/0034

(57) ABSTRACT

An ultrasound treatment device according to an embodiment of the present disclosure may include a scan ultrasound unit, a target generation unit, and a treatment ultrasound unit. The scan ultrasound unit may transmit a scan ultrasound transmission signal to scan the inside of a human body and receive a scan ultrasound reception signal reflected from the human body. The target generation unit may generate a target line based on the scan ultrasound reception signal. The treatment ultrasound unit may transmit a treatment ultrasound transmission signal along the target line. The ultrasound treatment device according to the present disclosure may transmit the treatment ultrasound transmission signal along the target line generated based on the scan ultrasound reception signal received from the inside of the human body, thereby further improving the treatment and cosmetic effect using ultrasound.

### BACKGROUND

### 1. Field

The present disclosure relates to an ultrasound treatment device.

### 2. Description of Related Art

Existing ultrasound treatment devices were capable of only treatment, making it difficult to know the progress of treatment.

In order to solve this problem, various studies capable of combining both treatment and monitoring have been conducted.

### RELATED ART DOCUMENT

### Patent Document

(Korean Registration Patent) No. 10-2642526 (Registration date, Feb. 26, 2024)

### SUMMARY

The present disclosure is directed to providing an ultrasound treatment device capable of further improving the treatment and cosmetic effect using ultrasound by transmitting a treatment ultrasound transmission signal along a target line generated based on a scan ultrasound reception signal received from the inside of the human body.

In order to solve the above problems, an ultrasound treatment device according to an embodiment of the present disclosure may include a scan ultrasound unit, a target generation unit, and a treatment ultrasound unit. The scan ultrasound unit may transmit a scan ultrasound transmission signal to scan the inside of a human body and receive a scan ultrasound reception signal reflected from the human body. The target generation unit may generate a target line based on the scan ultrasound reception signal. The treatment ultrasound unit may transmit a treatment ultrasound transmission signal along the target line.

In an embodiment, the ultrasound treatment device may further include an interval adjustment unit. The interval adjustment unit may adjust a transmission interval corresponding to a time interval for transmitting the scan ultrasound transmission signal.

In an embodiment, the target generation unit may further include a point generation unit. The point generation unit may generate a plurality of target points based on the scan ultrasound reception signals corresponding to the scan ultrasound transmission signals transmitted for each transmission interval.

In an embodiment, the ultrasound treatment device may further include a driving unit. The driving unit may move the scan ultrasound unit along the skin of the human body.

In an embodiment, each of the plurality of target points may be determined according to the magnitude of each of the scan ultrasound reception signals according to a depth from the skin of the human body.

In an embodiment, the target generation unit may further include a line generation unit. The line generation unit may generate the target line based on the plurality of target points.

In an embodiment, the line generation unit may further include a first line generation unit and a second line generation unit. The first line generation unit may generate a connection target line by sequentially connecting the plurality of target points. The second line generation unit may generate an average target line determined as an average value of depths from the skin of the human body corresponding to the plurality of target points.

In an embodiment, the target generation unit may further include a selection unit. The selection unit may select one of the connection target line and the average target line and provide the selected on as a target line.

In an embodiment, the selected target line may be moved up/down according to an input value.

In an embodiment, the ultrasound treatment device may further include a position determination unit. The position determination unit may determine a transmission position corresponding to a position at which the treatment ultrasound transmission signal is transmitted.

In an embodiment, when the target line is the average target line, if the inclination of the connection target line is greater than 0, the ultrasound treatment device may transmit the treatment ultrasound transmission signal to a position separated from the average target line by a predetermined distance in a first direction.

In addition to the technical problems of present disclosure described above, other features and advantages of present disclosure are described below, or can be clearly understood by those skilled in the art from such description and description.

### BRIEF DESCRIPTION OF DRAWINGS

FIG. 1 is a diagram illustrating an ultrasound treatment device according to embodiments of the present disclosure.

FIG. 2 is a diagram for describing the inside of a human body to which the ultrasound treatment device of FIG. 1 is applied.

FIGS. 3 to 5 are diagrams for describing an operation of a scan ultrasound unit included in the ultrasound treatment device of FIG. 1.

FIGS. 6 and 7 are diagrams for describing an operation of a point generation unit included in the ultrasound treatment device of FIG. 1.

FIGS. 8 and 9 are diagrams for describing an operation of a line generation unit included in the ultrasound treatment device of FIG. 1.

FIG. 10 is a diagram for describing an operation of a position determination unit included in the ultrasound treatment device of FIG. 1.

### DETAILED DESCRIPTION

In the specification, in adding reference numerals to components throughout the drawings, it is to be noted that like reference numerals designate like components even though components are shown in different drawings.

On the other hand, the meaning of the terms described in this specification should be understood as follows.

Singular expressions should be understood to include plural expressions unless the context clearly defines otherwise, and the scope of the rights should not be limited by these terms.

Also, it should be understood that terms such as “include” or “have” do not preclude the existence or addition possibility of one or more other features or numbers, steps, operations, components, parts, or combinations thereof.

Hereinafter, preferred embodiments of the present disclosure designed to solve the above problems will be described in detail with reference to the attached drawings.

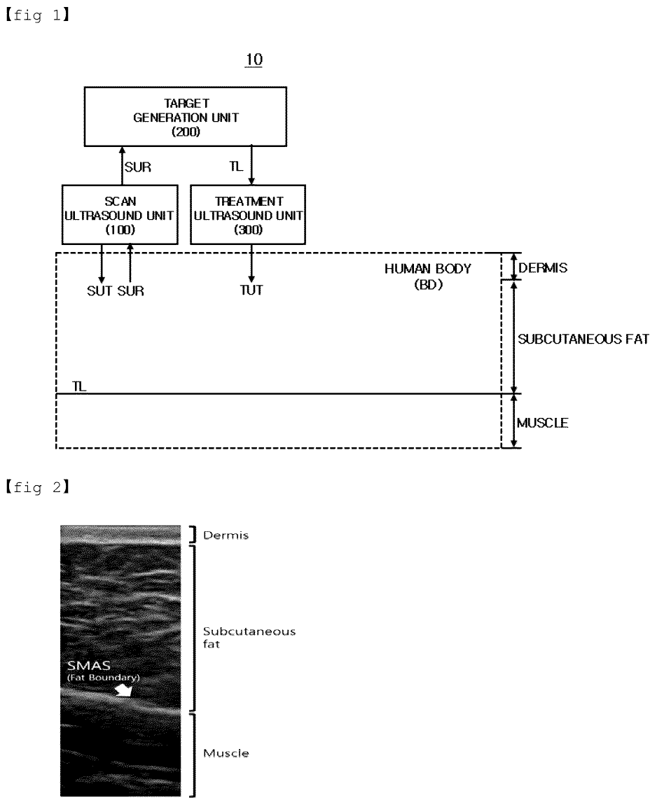

FIG. 1 is a diagram illustrating an ultrasound treatment device according to embodiments of the present disclosure. FIG. 2 is a diagram for describing the inside of a human body to which the ultrasound treatment device of FIG. 1 is applied.

Referring to FIGS. 1 and 2, an ultrasound treatment device 10 according to an embodiment of the present disclosure may include a scan ultrasound unit 100, a target generation unit 200, and a treatment ultrasound unit 300. The scan ultrasound unit 100 may transmit a scan ultrasound transmission signal SUT to scan the inside of a human body BD and receive a scan ultrasound reception signal SUR reflected from the human body BD. For example, the scan ultrasound unit 100 may transmit the scan ultrasound transmission signal SUT to the inside of the skin of the human body BD by using a single element and receive the scan ultrasound reception signal SUR reflected from the inside of the skin. The scan ultrasound unit 100 according to the present disclosure is not limited to the single element, but may include a plurality of elements.

The target generation unit 200 may generate a target line TL based on the scan ultrasound reception signal SUR. For example, the scan ultrasound reception signal SUR reflected from the inside of the skin may include information about a magnitude AM of the scan ultrasound reception signal SUR from the skin according to a depth DP. The target generation unit 200 according to the present disclosure may generate the target line TL by using the magnitude AM of the scan ultrasound reception signal SUR according to the depth DP of the skin.

The treatment ultrasound unit 300 may transmit a treatment ultrasound transmission signal TUT along the target line TL. For example, the treatment ultrasound unit 300 may transmit the treatment ultrasound transmission signal TUT along the target line TL generated from the target generation unit 200 by using a single element. The treatment ultrasound unit 300 according to the present disclosure is not limited to the single element, but may include a plurality of elements.

The inside of the human body BD may include a dermis layer, a subcutaneous fat layer, and a muscle layer. The fascia may be formed between the subcutaneous fat layer and the muscle layer, and when the treatment ultrasound transmission signal TUT is transmitted along the fascia, the skin care and treatment effects may be improved. For example, the target line TL generated by the target generation unit 200 according to the present disclosure may be the fascia corresponding to a boundary surface between the subcutaneous fat layer and the muscle layer.

The ultrasound treatment device 10 according to the present disclosure may transmit the treatment ultrasound transmission signal TUT along the target line TL generated based on the scan ultrasound reception signal SUR received from the inside of the human body BD, thereby further improving the treatment and cosmetic effect using ultrasound.

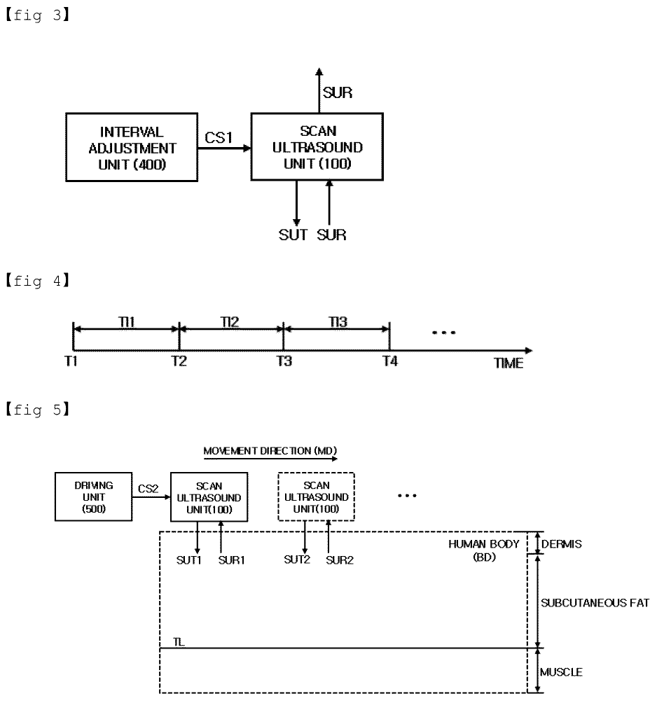

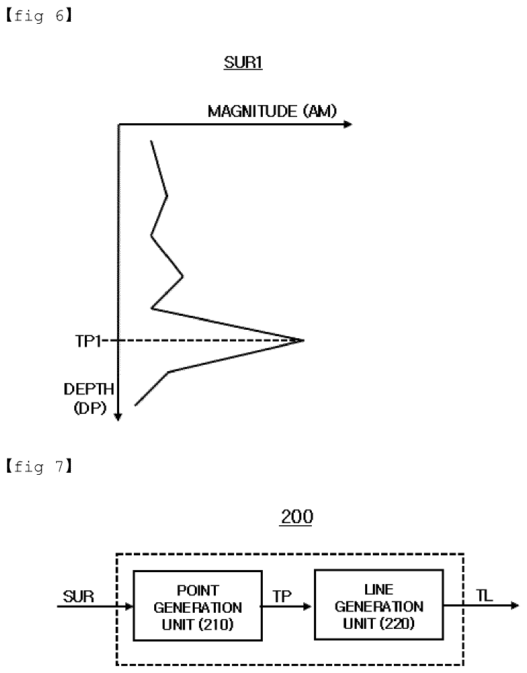

FIGS. 3 to 5 are diagrams for describing an operation of a scan ultrasound unit included in the ultrasound treatment device of FIG. 1.

Referring to FIGS. 1 to 5, in an embodiment, the ultrasound treatment device 10 may further include an interval adjustment unit 400. The interval adjustment unit 400 may adjust a transmission interval corresponding to a time interval for transmitting the scan ultrasound transmission signal SUT.A driving unit 500 may move the scan ultrasound unit 100 and the treatment ultrasound unit 300 along a skin surface. For example, a plurality of times may include a first time T1 to a fourth time T4. The scan ultrasound unit 100 may transmit a first scan ultrasound transmission signal SUT1 to the inside of the skin and receive a first scan ultrasound reception signal SUR1 reflected from the inside of the skin at the first time T1. The scan ultrasound unit 100 may move along the skin surface by the driving unit 500 during a first transmission interval between the first time T1 to a second time T2, and transmit a second scan ultrasound transmission signal SUT2 to the inside of the skin and receive a second scan ultrasound reception signal SUR2 reflected from the inside of the skin at the second time T2. In the same manner, the scan ultrasound unit 100 may receive a third scan ultrasound reception signal SUR3 and a fourth ultrasound reception signal.

In an embodiment, the ultrasound treatment device 10 may further include the driving unit 500. The driving unit 500 may move the scan ultrasound unit 100 along the skin of the human body BD. For example, the driving unit 500 may move the scan ultrasound unit 100 and the treatment ultrasound unit 300 along the skin surface in a movement direction MD based on a first control signal CS1, and the scan ultrasound unit 100 and the treatment ultrasound unit 300 may each include a mechanical device including a driving motor inside to enable the scan ultrasound unit 100 and the treatment ultrasound unit 300 to move.

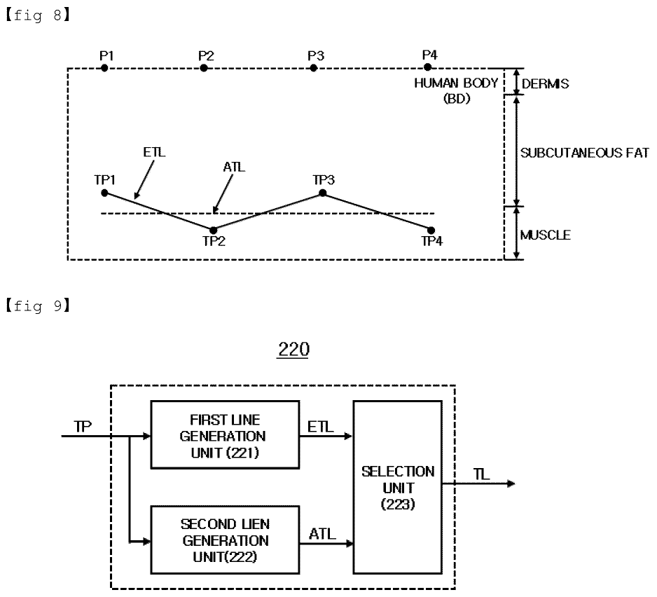

FIGS. 6 and 7 are diagrams for describing an operation of a point generation unit included in the ultrasound treatment device of FIG. 1.

Referring to FIGS. 1 to 7, in an embodiment, the target generation unit 200 may further include a point generation unit 210. The point generation unit 210 may generate a plurality of target points TP based on scan ultrasound reception signals SUR corresponding to the scan ultrasound transmission signals SUT transmitted for each transmission interval T1.

In an embodiment, each of the plurality of target points TP may be determined according to the magnitude AM of each of the scan ultrasound reception signals SUR according to the depth DP from the skin of the human body BD.

For example, a plurality of times may include the first time T1 to the fourth time T4. The scan ultrasound transmission signal SUT transmitted from the scan ultrasound unit 100 at the first time T1 may be the first scan ultrasound transmission signal SUT1, and the scan ultrasound reception signal SUR corresponding to the first scan ultrasound transmission signal SUT1 may be the first scan ultrasound reception signal SUR1. The first scan ultrasound reception signal SUR1 may include information about the magnitude AM of the scan ultrasound reception signal SUR according to the depth DP from the skin surface. The magnitude AM of the scan ultrasound reception signal SUR according to the depth DP from the skin surface with respect to the first scan ultrasound reception signal SUR1 may be illustrated as in FIG. 6. Here, the depth DP where the magnitude AM of the first scan ultrasound reception signal SUR1 is the greatest may be a first target point TP1.

In the same manner, the depth DP where the magnitude AM of the second scan ultrasound reception signal SUR2 is the greatest may be a second target point TP2, the depth DP where the magnitude AM of the third scan ultrasound reception signal SUR3 is the greatest may be a third target point TP3, and the depth DP where the magnitude AM of a fourth scan ultrasound reception signal SUR4 is the greatest may be a fourth target point TP4.

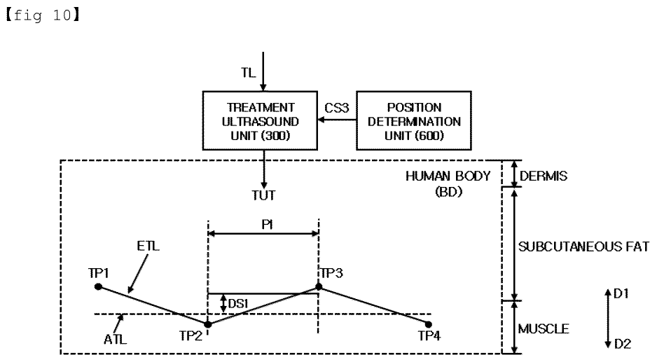

FIGS. 8 and 9 are diagrams for describing an operation of a line generation unit included in the ultrasound treatment device of FIG. 1.

Referring to FIGS. 1 to 9, in an embodiment, the target generation unit 200 may further include a line generation unit 220. The line generation unit 220 may generate the target line TL based on the plurality of target points TP.

In an embodiment, the line generation unit 220 may further include a first line generation unit 221 and a second line generation unit 222. For example, the plurality of target points TP may include the first target point TP1 to the fourth target point TP4. At the first time T1, the scan ultrasound unit 100 may transmit the first scan ultrasound transmission signal SUT1 and receive the first scan ultrasound reception signal SUR1 at a first position P1. In this case, the target point TP generated by the point generation unit 210 may be the first target point TP1. In addition, at the second time T2, the scan ultrasound unit 100 may move from the first position P1 to a second position P2 to transmit the second scan ultrasound transmission signal SUT2 and receive the second scan ultrasound reception signal SUR2. In this case, the target point TP generated by the point generator 210 may be the second target point TP2. In the same manner, the point generation unit 210 may generate the third target point TP3 and the fourth target point TP4.

The first line generation unit 221 may generate a connection target line ETL by sequentially connecting the plurality of target points TP. For example, the plurality of target points TP may include the first target point TP1 to the fourth target point TP4. The first line generation unit 221 may generate the connection target line ETL by sequentially connecting the first target point TP1 to the fourth target point TP4 generated in chronological order from the first time T1 to the fourth time T4.

The second line generation unit 222 may generate an average target line ATL determined as an average value of depths DP from the skin of the human body BD corresponding to the plurality of target points TP. For example, the plurality of target points TP may include the first target point TP1 to the fourth target point TP4. The second line generation unit 222 may generate a line corresponding to the average value of depths DP from the skin surface corresponding to each of the first target point TP1 to the fourth target point TP4 as the average target line ATL.

In an embodiment, the target generation unit 200 may further include a selection unit 223. The selection unit 223 may select one of the connection target line ETL and the average target line ATL and provide the selected one as the target line TL. For example, the selection unit 223 may select one of the connection target line ETL and the average target line ATL as the target line TL according to a user input and provide the selected one.

FIG. 10 is a diagram for describing an operation of a position determination unit included in the ultrasound treatment device of FIG. 1.

Referring to FIGS. 1 to 10, in an embodiment, the ultrasound treatment device 10 may further include a position determination unit 600. The position determination unit 600 may determine a transmission position corresponding to a position at which the treatment ultrasound transmission signal TUT is transmitted. For example, the treatment ultrasound unit 300 may include a mechanical device including a drive motor capable of moving a single element up and down, and control the single element up and down according to the transmission position determined by the position determination unit 600. In addition, when the treatment ultrasound unit 300 includes a plurality of elements, the treatment ultrasound unit 300 may control the transmission position by performing beam focusing by using the plurality of elements.

In an embodiment, when the target line TL is the average target line ATL, if the inclination of the connection target line ETL is greater than 0, the ultrasound treatment device 10 may transmit the treatment ultrasound transmission signal TUT to a position separated from the average target line ATL by a predetermined distance in a first direction D1.

For example, as shown in FIG. 10, in a period from the second target point TP2 to the third target point TP3, the inclination of the connection target line ETL connecting the second target point TP2 to the third target point TP3 may be greater than 0. In this case, the ultrasound treatment device 10 according to the present disclosure may transmit the treatment ultrasound transmission signal TUT along a line separated from the average target line ATL by a first distance DS1 in the first direction D1.

In addition, for example, as shown in FIG. 10, in a period from the third target point TP3 to the fourth target point TP4, the inclination of the connection target line ETL connecting the third target point TP3 to the fourth target point TP4 may be less than 0. In this case, the ultrasound treatment device 10 according to the present disclosure may transmit the treatment ultrasound transmission signal TUT along a line separated from the average target line ATL by the first distance DS1 in a second direction D2 corresponding to a direction opposite to the first direction D1.

In an embodiment, the first line generation unit 221 may move the position of the connection target line ETL in the human body up and down according to a first input value of a user and provide the moved position, and the second line generation unit 222 may move the position of the average target line ATL in the human body up and down according to a second input value of the user and provide the moved position. The ultrasound treatment device 10 according to the present disclosure may adjust the intensity and interval of the treatment ultrasound transmission signal TUT, or a movement speed of the treatment ultrasound unit 300 based on the depths of the plurality of target points TP and the treatment intensity according to a previously input type of a treatment skin layer.

The ultrasound treatment device 10 according to the present disclosure may transmit the treatment ultrasound transmission signal TUT along the target line TL generated based on the scan ultrasound reception signal SUR received from the inside of the human body BD, thereby further improving the treatment and cosmetic effect using ultrasound.

According to present disclosure as described above, the following effects are provided.

The ultrasound treatment device according to the present disclosure may transmit a treatment ultrasound transmission signal along a target line generated based on a scan ultrasound reception signal received from the inside of the human body, thereby further improving the treatment and cosmetic effect using ultrasound.

In addition, other features and advantages of present disclosure may be newly identified through embodiments of present disclosure.

In addition to the technical problems of present disclosure described above, other features and advantages of present disclosure are described below, or can be clearly understood by those skilled in the art from such description and description.

What is claimed is:

1. An ultrasound treatment device comprising:
a scan ultrasound unit configured to transmit a scan ultrasound transmission signal to scan an inside of a human body and receive a scan ultrasound reception signal reflected from the human body;
a target generation unit configured to generate a target line based on the scan ultrasound reception signal; and
a treatment ultrasound unit configured to transmit a treatment ultrasound transmission signal along the target line.

2. The ultrasound treatment device of claim 1, further comprising: an interval adjustment unit configured to adjust a transmission interval corresponding to a time interval for transmitting the scan ultrasound transmission signal.

3. The ultrasound treatment device of claim 2, wherein the target generation unit further includes a point generation unit configured to generate a plurality of target points based on the scan ultrasound reception signals corresponding to the scan ultrasound transmission signals transmitted for each transmission interval.

4. The ultrasound treatment device of claim 3, further comprising: a driving unit configured to move the scan ultrasound unit along a skin of the human body.

5. The ultrasound treatment device of claim 4, wherein each of the plurality of target points is determined according to a magnitude of each of the scan ultrasound reception signals according to a depth from the skin of the human body.

6. The ultrasound treatment device of claim 5, wherein the target generation unit further includes a line generation unit configured to generate the target line based on the plurality of target points.

7. The ultrasound treatment device of claim 6, wherein the line generation unit further includes:
a first line generation unit configured to generate a connection target line by sequentially connecting the plurality of target points; and
a second line generation unit configured to generate an average target line determined as an average value of depths from the skin of the human body corresponding to the plurality of target points.

8. The ultrasound treatment device of claim 7, wherein the target generation unit further includes a selection unit configured to select one of the connection target line and the average target line and provide the selected on as a target line.

9. The ultrasound treatment device of claim 8, further comprising: a position determination unit configured to determine a transmission position corresponding to a position at which the treatment ultrasound transmission signal is transmitted.

10. The ultrasound treatment device of claim 9, wherein when the target line is the average target line, if an inclination of the connection target line is greater than 0, the ultrasound treatment device is configured to transmit the treatment ultrasound transmission signal to a position separated from the average target line by a predetermined distance in a first direction.

11. The ultrasound treatment device of claim 7, wherein the first line generation unit is configured to move the connection target line up and down according to a first input value of a user and provide the moved connection target line.

12. The ultrasound treatment device of claim 7, wherein the second line generation unit is configured to move the average target line up and down according to a second input value of a user and provide the moved average target line.

13. The ultrasound treatment device of claim 8, wherein the ultrasound treatment device is configured to adjust an intensity and interval of the treatment ultrasound transmission signal, or a movement speed of the treatment ultrasound unit based on depths of the plurality of target points and a treatment intensity according to a previously input type of a treatment skin layer.
